(19) United States
(12) Patent Application Publication
(10) Pub. No.: US 2020/0126644 A1
(43) Pub. Date: Apr. 23, 2020

(54) APPLYING MACHINE LEARNING TO SCRIBE INPUT TO IMPROVE DATA ACCURACY

(71) Applicant: 3M INNOVATIVE PROPERTIES COMPANY, St. Paul, MN (US)

(72) Inventor: Thomas S. Polzin, Pittsburgh, PA (US)

(21) Appl. No.: 16/661,251

(22) Filed: Oct. 23, 2019

Related U.S. Application Data
(60) Provisional application No. 62/749,431, filed on Oct. 23, 2018.

(51) Int. Cl. G16H 10/60; G06N 20/00; G10L 17/00; G10L 17/06

(52) U.S. Cl. CPC G16H 10/60; G10L 17/06; G10L 17/005; G06N 20/00

(57) ABSTRACT

A computerized system learns a mapping from the speech of a physician and patient in a physician-patient encounter to discrete information to be input into the patient's Electronic Medical Record (EMR). The system learns this mapping based on a transcript of the physician-patient dialog, an initial state of the EMR (before the EMR was updated based on the physician-patient dialogue), and a final state of the EMR (after the EMR was updated based on the physician-patient dialog). The learning process is enhanced by taking advantage of knowledge of the differences between the initial EMR state and the final EMR state.

### BACKGROUND

When a physician or other healthcare professional provides healthcare services to a patient or otherwise engages with a patient in a patient encounter, the healthcare professional typically creates documentation of that encounter. For example, healthcare providers often engage human medical scribes, who listen to a physician-patient dialogue while the patient's electronic medical record (EMR) is open in front of them on a computer screen. It is the task of the medical scribe to map the dialogue into discrete information, input it into the respective EMR system, and create a clinical report of the physician-patient encounter. The process can be labor-intensive and prone to error.

### SUMMARY

A computerized system learns a mapping from the speech of a physician and patient in a physician-patient encounter to discrete information to be input into the patient's Electronic Medical Record (EMR). The system learns this mapping based on a transcript of the physician-patient dialog, an initial state of the EMR (before the EMR was updated based on the physician-patient dialogue), and a final state of the EMR (after the EMR was updated based on the physician-patient dialog). The learning process is enhanced by taking advantage of knowledge of the differences between the initial EMR state and the final EMR state.

One aspect of the present invention is directed to a method performed by at least one computer processor executing computer program instructions tangibly stored on at least one non-transitory computer-readable medium. The method includes, at a transcription job routing engine: (A) saving an initial state of an electronic medical record (EMR) of a first person; (B) saving a final state of the EMR of the first person after the EMR of the first person has been modified based on speech of the first person and speech of a second person; (C) identifying differences between the initial state of the EMR of the third person and the final state of the EMR of the third person; and (D) applying a machine learning module to: (D)(1) a transcript of the speech of the first person and the speech of the second person; and (D)(2) the differences between the initial state of the EMR of the first person and the final state of the EMR of the first person, to generate a mapping between: (a) the transcript of the speech of the first person and the speech of the second person; and (b) the differences between the initial state of the EMR and the final state of the EMR.

The method may further include, before (B): (E) capturing the speech of the first person and the speech of a second person to produce at least one audio signal representing the speech of the first person and the speech of the second person; and (F) applying automatic speech recognition to the at least one audio signal to produce the transcript of the speech of the first person and the speech of the second person. The method may further include, before (B): (G) identifying an identity of the first person; (H) identifying an identity of the second person; and wherein (F) comprises producing the transcript of the speech of the first person and the speech of the second person based on the identity of the first person, the identity of the second person, and the speech of the first person and the speech of the second person. (F) may further include associating the identity of the first person with a first portion of the transcript and associating the identity of the second person with a second portion of the transcript.

Step (A) may include converting the initial state of the EMR into a text file.

Step (A) may include converting the initial state of the EMR of the first person into a list of discrete medical domain model instances.

Step (B) may include converting the final state of the EMR of the first person into a text file.

Step (B) may include converting the final state of the EMR of the first person into a list of discrete medical domain model instances.

Step (C) may include using non-linear alignment techniques to identify the differences between the initial state of the EMR of the first person and the final state of the EMR of the first person.

The method may further include: (E) saving an initial state of an electronic medical record (EMR) of a third person; (F) saving a final state of the EMR of the third person after the EMR of the third person has been modified based on speech of the third person and speech of a fourth person; (G) identifying differences between the initial state of the EMR of the third person and the final state of the EMR of the third person; and (H) applying a machine learning module to: (1) the transcript of the speech of the first person and the speech of the second person; (2) the differences between the initial state of the EMR of the first person and the final state of the EMR of the first person; (3) the transcript of the speech of the third person and the speech of the fourth person; and (4) the differences between the initial state of the EMR of the third person and the final state of the EMR of the third person; thereby generating a mapping between text and EMR state differences.

Another aspect of the present invention is directed to a system comprising at least one non-transitory computer-readable medium having computer program instructions stored thereon for causing at least one computer processor to perform a method. The method includes, at a transcription job routing engine: (A) saving an initial state of an electronic medical record (EMR) of a first person; (B) saving a final state of the EMR of the first person after the EMR of the first person has been modified based on speech of the first person and speech of a second person; (C) identifying differences between the initial state of the EMR of the third person and the final state of the EMR of the third person; and (D) applying machine learning to: (1) a transcript of the speech of the first person and the speech of the second person; and (2) the differences between the initial state of the EMR of the first person and the final state of the EMR of the first person, to generate a mapping between: (a) the transcript of the speech of the first person and the speech of the second person; and (b) the differences between the initial state of the EMR and the final state of the EMR.

Other features and advantages of various aspects and embodiments of the present invention will become apparent from the following description and from the claims.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a dataflow diagram of a system for generating training data for a supervised machine learning module to map from speech of a physician and speech of a patient to a final state of an Electronic Medical Record (EMR) of the patient according to one embodiment of the present invention.

FIG. 2 is a flowchart of a method 200 performed by the system of FIG. 1 according to one embodiment of the present invention.

FIG. 3 is a dataflow diagram of a system for performing supervised learning on training data to learn a mapping from a transcript to the differences between an initial EMR state and a final EMR state according to one embodiment of the present invention.

FIG. 4 is a flowchart of a method performed by the system of FIG. 3 according to one embodiment of the present invention.

FIG. 5 is a diagram illustrating encodings of an initial EMR state, and an internal hidden layer in a machine learning model according to one embodiment of the present invention.

### DETAILED DESCRIPTION

As described above, when a physician or other healthcare professional provides healthcare services to a patient or otherwise engages with a patient in a patient encounter, the healthcare professional typically creates documentation of that encounter (such as in the form of a clinical note), or a medical scribe may assist in creating the documentation, either by being in the room or listening to the encounter in real time via a remote connection or by listening to a recording of the encounter. This removes some of the burden of a typical workflow of many physicians, by taking step (3) of a typical physician workflow, below, out of the physician's responsibilities, and having the medical scribe perform that work, so that the physician can focus on the patient during the physician-patient encounter. A typical physician workflow when treating patients is the following:
(1) Prepare for the patient visit by reviewing information about the patient in an Electronic Medical Record (EMR) system.
(2) Engage in the patient encounter, such as by:
   a. Meeting with the patient in a treatment room.
   b. Discussing, with the patient, the reason for the visit, any changes in the patient's health conditions, medications, etc.
   c. Examining the patient.
   d. Discussing the physician's findings and plan with the patient.
   e. Entering any required follow up actions and medication orders into the EMR.
(3) Create a clinical report of the patient encounter, containing information such as the care provided to the patient and the physician's treatment plan, such as by any one or more of the following:
   a. Writing or speaking a free form text narrative, beginning with a blank editing screen. If the physician speaks, the physician's speech may be transcribed verbatim into the editing screen using automatic speech recognition (ASR) software.
   b. Starting with a document template containing partial content, such as section headings and partially completed sentences, and filling in the missing information from the patient encounter, whether by typing or speaking, to create a clinical note for the physician-patient encounter.
   c. Using a structured data entry user interface to enter discrete data elements and free form text into the patient's EMR, such as by selecting discrete data elements using buttons and drop-down lists, and typing or speaking free form text into text fields.

In general, embodiments of the present invention include computerized systems and methods which learn how to update a patient's EMR automatically, based on transcripts of physician-patient encounters and the corresponding EMR updates that were made based on those transcripts. As a resulting of this learning, the work required to update EMRs based on physician-patient encounters may be partially or entirely eliminated.

Furthermore, embodiments of the present do not merely automate the work that previously was performed by a physician, scribe, and other humans. Instead, embodiments of the present invention include computer-automated methods and systems which update patients' EMRs automatically using techniques that are fundamentally different than those currently used by humans to update EMRs. These techniques, which involve the use of machine learning applied to a transcript of the physician-patient dialog and states of the EMR before and after the EMR was updated based on the physician-patient dialogue, are inherently rooted in computer technology and, when implemented in computer systems and methods, result in an improvement to computer technology in the form of a computer that is capable of automatically updating patient EMRs in a way that both improves the quality of the EMR and that was not previously used (by humans or otherwise) to update EMRs.

One problem addressed and solved by embodiments of the present invention is the problem of how to update a computer-implemented EMR to reflect the content of a physician-patient dialog automatically (i.e., without human interaction). A variety of ways in which embodiments of the present invention solve this problem through the use of computer-automated systems and methods will now be described.

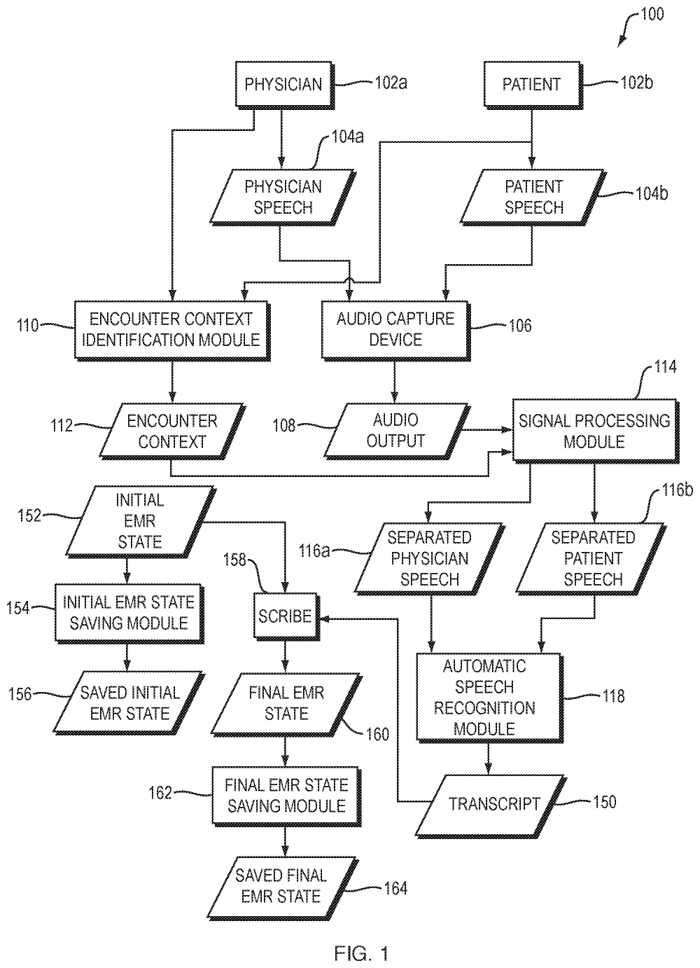

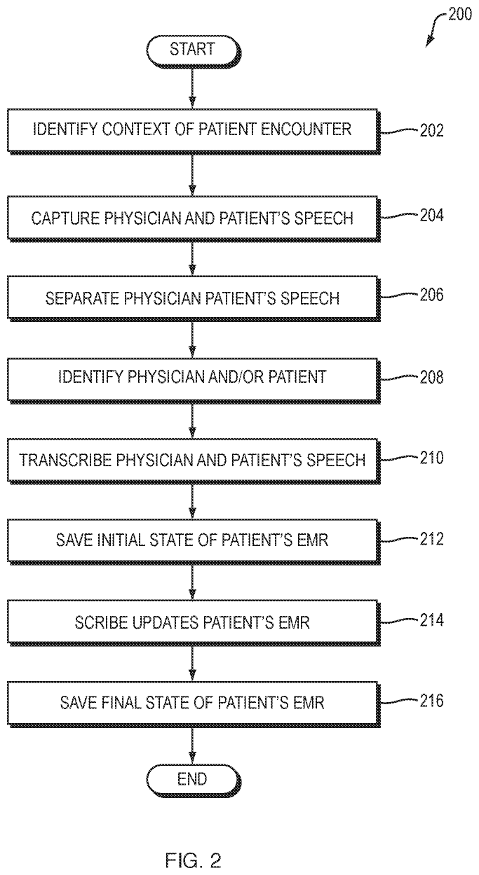

Referring to FIG. 1, a dataflow diagram is shown of a system 100 for automatically generating a clinical report 150 (also referred to herein as a “transcript”) of an encounter between a physician 102a and a patient 102b according to one embodiment of the present invention. Referring to FIG. 2, a flowchart is shown of a method 200 performed by the system 100 of FIG. 1 according to one embodiment of the present invention.

The system 100 includes a physician 102a and a patient 102b. More generally, the system 100 may include any two or more people. For example, the role played by the physician 102a in the system 100 may be played by any one or more people, such as one or more physicians, nurses, radiologists, or other healthcare providers, although embodiments of the present invention are not limited to use in connection with healthcare providers. Similarly, the role played by the patient 102b in the system 100 may be played by any one or more people, such as one or more patients and/or family members, although embodiments of the present invention are not limited to use in connection with patients. The physician 102a and patient 102b may, but need not, be in the same room as each other or otherwise in physical proximity to each other. The physician 102a and patient 102b may instead, for example, be located remotely from each other (e.g., in different rooms, buildings, cities, or countries) and communicate with each other by telephone/videoconference and/or over the Internet or other network.

The system 100 also includes an encounter context identification module 110, which identifies and/or generates encounter context data 112 representing properties of the physician-patient encounter (FIG. 2, operation 202). The encounter context identification module 110 may, for example, generate the encounter context data 112 based on information received from the physician 102a and/or the patient 102b or an EMR. For example, the physician 102a may explicitly provide input representing the identity of the physician 102a and/or patient 102b to the encounter context identification module 110. The encounter context identification module 110 may generate the encounter context data 112 using speaker identification/verification techniques. As an example of speaker verification techniques, a user may provide credentials to a log-in user interface (not shown), which the system 100 may use to identify the speaker; the system 100 may also optionally verify that the speaker is authorized to access the system 100. As another example, the user may provide credentials via a speech-based speaker verification system. As another example, the patient 102b may explicitly provide input representing the identity of the physician 102a and/or patient 102b to the encounter context identification module 110. As another example, the encounter context identification module 110 may identify the patient 102b based on data from another system, such as an EMR or a scheduling system which indicates that the patient 102b is scheduled to see the physician 102a at the current time.

Regardless of how the encounter context identification module 110 generates the encounter context data 112, the encounter context data 112 may, for example, include data representing any one or more of the following, in any combination:
Patient context data representing information about the patient 102b that is known before the patient encounter, such as any one or more of the following, in any combination:
   The identity of the patient 102b.
   Demographic information about the patient 102b, such as gender and age.
   Medical information about the patient 102b, such as known past or current problems, especially major health problems (e.g., cancer) or chronic conditions, current and past medications, allergies, and recent lab values.
Physician context data representing information about the physician 102a, such as any one or more of the following, in any combination:
   The identity of the physician 102a.
   The medical specialty and setting of care of the physician 102a.
   Explicit preferences of the physician 102a, such as document templates to be used, macro expansions (e.g., identifying a preference for expressing routine findings, such as “.nfc” that should be expanded to “No fever or chills”), rules for documenting specific procedures, and typing guidelines (e.g., which terms to abbreviate and which terms to spell out fully).
   Implicit preferences of the physician 102a, which may be derived automatically by the system 100 based on previous clinical reports associated with the physician 102a, such as verbosity and word choice of the physician 102a.
Patient encounter context, such as the reason for the visit, e.g., the patient 102b's chief complaint, the location of the encounter, and the type of the encounter (e.g., well visit, follow up after a procedure, scheduled visit to monitor a chronic condition).
Work in progress data, such as any one or more of the following, in any combination:
   A partial clinical report 150 for the patient encounter, including the text of the note, the current cursor position in the note, and the left and right textual context of the cursor in the note.
   The output of a natural language understanding subsystem for classifying and/or encoding the semantic content of the partially generated clinical report 150 as it is being typed.
   The output of a dialog processing system (e.g., module 118).

Now assume that the physician 102a and patient 102b speak during the physician 102a's encounter with the patient 102b. The physician's speech 104a and patient's speech 104b are shown as elements of the system 100. The physician 102a's speech 104a may, but need not be, directed at the patient 102b. Conversely, the patient 102b's speech 104b may, but need not be, directed at the physician 102a. The system 100 includes an audio capture device 106, which captures the physician's speech 104a and the patient's speech 104b, thereby producing audio output 108 (FIG. 2, operation 204). The audio capture device 106 may, for example, be one or more microphones, such as a microphone located in the same room as the physician 102a and the patient 102b, or distinct microphones spoken into by the physician 102a and the patient 102b. In the case of multiple audio capture devices, the audio output may include multiple audio outputs, which are shown as the single audio output 108 in FIG. 1 for ease of illustration.

The audio output 108 may, for example, contain only audio associated with the patient encounter. This may be accomplished by, for example, the audio capture device 106 beginning to capture the physician and patient speech 104a-b at the beginning of the patient encounter and terminating the capture of the physician and patient speech 104a-b at the end of the patient encounter. The audio capture device 106 may identify the beginning and end of the patient encounter in any of a variety of ways, such as in response to explicit input from the physician 102a indicating the beginning and end of the patient encounter (such as by pressing a “start” button at the beginning of the patient encounter and an “end” button at the end of the patient encounter). Even if the audio output 108 contains audio that is not part of the patient encounter, the system 100 may crop the audio output 108 to include only audio that was part of the patient encounter.

The system 100 may also include a signal processing module 114, which may receive the audio output 108 as input, and separate the audio output 108 into separate audio signals 116a and 116b representing the speech 104a of the physician 102a and the speech 104b of the patient 102b, respectively (FIG. 2, operation 206). The signal processing module 114 may use any of a variety of signal source separation techniques to produce the separated physician speech 116a and the separated patient speech 116b, which may or may not be identical to the original physician speech 104a and patient speech 104b, respectively. Instead, the separated physician speech 116a may be an estimate of the physician speech 104a and the separated patient speech 116b may be an estimate of the patient speech 104b.

The separated physician speech 116a and separated patient speech 116b may contain more than just audio signals representing speech. For example, the signal processing module 114 may identify the physician 102a (e.g., based on the audio output 108 and/or the encounter context data 112) and may include data representing the identity of the physician 102a in the separated physician speech 116a. Similarly, the signal processing module 114 may identify the patient 102b (e.g., based on the audio output 108 and/or the encounter context data 112) and may include data representing the identity of the patient 102b in the separated patient speech 116b (FIG. 2, operation 208). The signal processing module 114 may use any of a variety of speaker clustering, speaker identification, and speaker role detection techniques to identify the physician 102a and patient 102b and their respective roles (e.g., physician, nurse, patient, parent, caretaker).

The system 100 also includes an automatic speech recognition (ASR) module 118, which may use any of a variety of known ASR techniques to produce a transcript 150 of the physician speech 116a and patient speech 116b (FIG. 2, operation 210). The transcript 150 may include text representing some or all of the physician speech 116a and patient speech 116b, which may be organized within the transcript 150 in any of a variety of ways. For example, the transcript 150 may include data (e.g., text and/or markup) associating the physician 102a with corresponding text transcribed from the physician speech 116a, and may include data (e.g., text and/or markup) associating the patient 102b with corresponding text transcribed from the patient speech 116b. As a result, the speaker (e.g., the physician 102a or the patient 102b) who spoke any part of the text in the transcript 150 may be easily identified based on the identification data in the transcript 150.

The system 100 may identify the patient's EMR. The state of the patient's EMR before the EMR is modified (e.g., by the scribe) based on the physician speech 116a, patient speech 116b, or the transcript 150 is referred to herein as the “initial EMR state” 152. The system 100 includes an initial EMR state saving module 154, which saves the initial EMR state 152 as a saved EMR state 156. The EMR state saving module 154 may, for example, convert the initial EMR state 152 into text and save that text in a text file, or convert the initial EMR state 152 into a list of discrete medical domain model instances (e.g., Fast Healthcare Interoperability Resources (FHIR)) (FIG. 2, operation 212). The process of saving the saved initial EMR state 156 may include, for example, extracting, modifying, summarizing, converting, or otherwise processing some or all of the initial EMR state 152 to produce the saved initial EMR state 156.

The scribe 158 updates the patient's EMR in the normal manner, such as based on the transcript 150 of the physician-patient dialog, the physician speech 102a, and/or the patient speech 102b (FIG. 2, operation 214). The resulting updated EMR has a state that is referred to herein as the “final EMR state” 160. The scribe 158 may update the patient's EMR in any of a variety of well-known ways, such as by identifying a finding, diagnosis, medication, prognosis, allergy, or treatment in the transcript 150 and updating the initial EMR state 152 to reflect that finding, diagnosis, medication, prognosis, allergy, or treatment within the final EMR state 160.

The system 100 includes a final EMR state saving module 162, which saves the final EMR state 160 as a saved final EMR state 164. The EMR state saving module 162 may, for example, convert the final EMR state 160 into text and save that text in a text file, or convert the final EMR state 160 into a list of discrete medical domain model instances (e.g., FHIR) (FIG. 2, operation 216). The final EMR state saving module 162 may, for example, generate the saved final EMR state 164 based on the final EMR state 160 in any of the ways disclosed above in connection with the generation of the saved initial EMR state 156 by the initial EMR state saving module 154.

At this point, the system 100 includes three relevant units of data (e.g., documents): the transcript 150 of the physician-patient dialog, the saved initial EMR state 156, and the saved final EMR state 164. Note that the creation of these documents need not impact the productivity of the scribe 158 compared to existing processes. For example, even if the transcript 150, saved initial EMR state 156, and saved final EMR state 164 are not saved automatically, the scribe 168 may save them with as little as one mouse click each.

As will now be described in more detail, the transcript 150, saved initial EMR state 156, and saved final EMR state 164 may be used as training data to train a supervised machine learning algorithm. Embodiments of the present invention are not limited to use in connection with any particular machine learning algorithm. Examples of supervised machine learning algorithms that may be used in connection with embodiments of the present invention include, but are not limited to, support vector machines, linear regression algorithms, logistic regression algorithms, naive Bayes algorithms, linear discriminant analysis algorithms, decision tree algorithms, k-nearest neighbor algorithms, neural networks, and similarity learning algorithms.

More training data may be generated and used to train the supervised machine learning algorithm by repeating the process described above for a plurality of additional physician-patient dialogues. Such dialogues may involve the same or different patient. If they involve different patients, then the corresponding EMRs may be different than the EMR of the patient 102b. As a result, the training data that is used to train the supervised machine learning algorithm may include training data corresponding to any number of physician-patient dialogs involving any number of patients and any number of corresponding EMRs.

In general, and as will be described in more detail below, the use of both the saved initial EMR state 156 and the saved final EMR state 164, instead of using only the saved final EMR state 164, simplifies the complexity of mapping the physician-patient dialogue transcript 150 to the final EMR state 164 significantly, because instead of trying to learn a mapping directly from the transcript 150 to the final EMR state 164, the system 100 only has to learn a mapping from the transcript 150 to the differences between the initial EMR state 156 and the final EMR state 164, and such differences will, in practice, be much simpler than the final EMR state 164 as a whole.

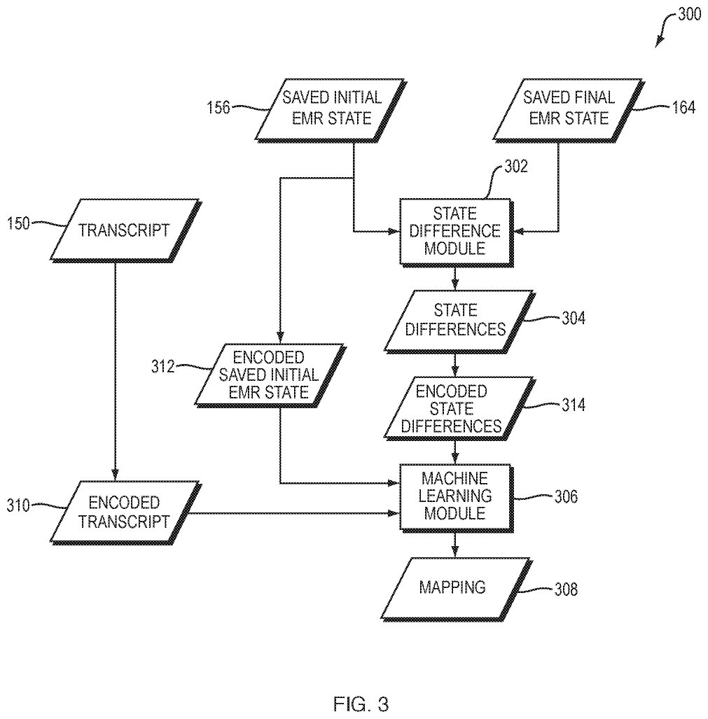

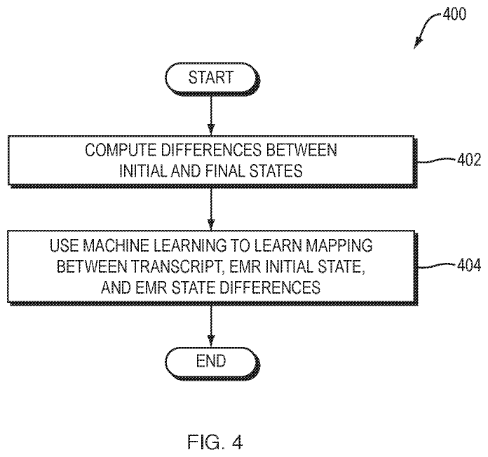

Referring to FIG. 3, a dataflow diagram is shown of a system 300 for performing supervised learning on the training data to learn a mapping from the transcript 150 to the differences between the initial EMR state 156 and the final EMR state 164 according to one embodiment of the present invention. Referring to FIG. 4, a flowchart is shown of a method 400 performed by the system 300 of FIG. 3 according to one embodiment of the present invention.

The system 300 includes a state difference module 302, which receives the initial EMR state 156 and final EMR state 164 as input, and which computes the differences of those states using, for example, non-linear alignment techniques, to produce as output a set of differences 304 of the two states 156 and 165 (FIG. 4, operation 402). Such differences 304 may be computed for any number of corresponding initial and final EMR states. These differences 304, in an appropriate representation, define the targets to be learned by a machine learning module 306. The machine learning module 306 receives as input the saved initial EMR state 156 (or an encoded saved initial EMR state 312, as described below) and corresponding pairs of transcripts (which may or may not be encoded, as described below) and corresponding EMR state differences (which may or may not be encoded, as described below), such as the transcript 150 and corresponding state differences 304. The state differences 304 define the expected output for use in the training performed by the machine learning module 306. Based on these inputs, the machine learning module 306 may use any of a variety of supervised machine learning techniques to learn a mapping 308 between the received inputs (FIG. 4, operation 404).

FIG. 3 shows that the machine learning module 306 receives as input the encoded transcript 310, encoded saved initial EMR state 312, and encoded state differences 314. Alternatively, however, the machine learning module 306 may receive as input the unencoded versions of one or more of those inputs. For example, the machine learning module may receive as input: (1) the transcript 150 or the encoded transcript 310; (2) the saved initial EMR state 156 or the encoded saved initial EMR state 312; and (3) the state differences 304 or the encoded state differences 314, in any combination. Therefore, any reference herein to the machine learning module 306 receiving as input one of the unencoded inputs (e.g., transcript 150, saved initial EMR state 156, or state differences 304) should be understood to refer equally to the corresponding encoded input (e.g., encoded transcript 310, encoded saved initial EMR state 312, or encoded state differences, respectively), and vice versa.

Once the mapping 308 has been generated, the mapping 308 may be applied to subsequent physician-patient transcripts to predict the EMR state changes that need to be made to an EMR based on those transcripts. For example, upon generating such a transcript, embodiments of the present invention may identify the current (initial) state of the patient's EMR, and then apply the mapping 308 to the identified initial state to identify state changes to apply to the patient's EMR. Embodiments of the present invention may then apply the identified state changes to update the patient's EMR accordingly and automatically, thereby eliminating the need for a human to manually make such updates, with the possible exception of human approval of the automatically-applied changes.

As described above, the mapping 308 may be generated based on one or more physician-patient dialogues and corresponding EMR state differences. Although the quality of the mapping 308 generally improves as the number of physician-patient dialogues and corresponding EMR state differences that are used to train the mapping 308 increases, in many cases the mapping 308 may be trained to a sufficiently high quality based on only a small number of physician-patient dialogues and corresponding EMR state differences. Embodiments of the present invention may, therefore, train and use an initial version of the mapping 308 in the ways described above based on a relatively small number of physician-patient dialogues and corresponding EMR state differences. This enables the mapping 308 to be applied to subsequent physician-patient dialogues, and to achieve the benefits described above, as quickly as possible. Then, as the systems 100 and 300 obtain additional physician-patient dialogues and corresponding EMR state differences, the systems 100 and 300 may use that additional data to further train the mapping 308 and thereby improve the quality of the mapping 308. The resulting updated mapping 308 may then be applied to subsequent physician-patient dialogues. This process of improving the mapping 308 may be repeated any number of times. In this way, the benefits of embodiments of the present invention may be obtained quickly, without waiting for a large volume of training data, and as additional training data becomes available, that data may be used to improve the quality of the mapping 308 repeatedly over time.

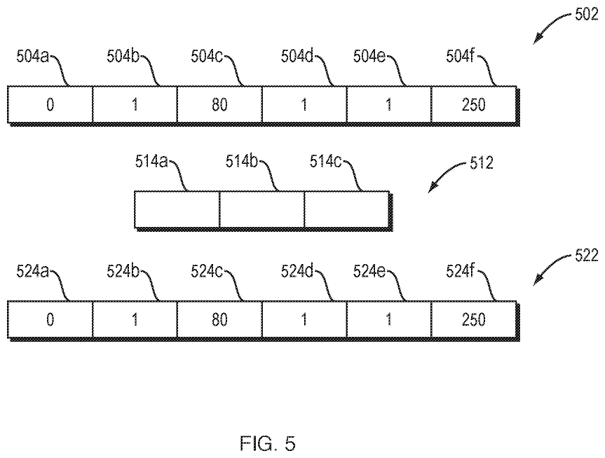

As described above, the initial EMR state saving module 154 may save the initial EMR state 152 as the saved initial EMR state 156, and the final EMR state saving module 162 may save the final EMR state 160 as the saved final EMR state 164. The saved initial EMR state 156 may be encoded in any of a variety of ways, such as in the manner shown in FIG. 5.

FIG. 5 shows input 522 and output 502 which may be used to train an autoencoder according to one embodiment of the present invention. The input 522 may, for example, implement the saved initial EMR state. For ease of illustration and explanation, in the example of FIG. 5, the input 522 encodes the saved initial EMR state 156 into a vector as follows:
A cell 524a contains binary data indicating whether the EMR indicates that the patient has a peanut allergy. In this example, the value of 0 indicates that the patient does not have a peanut allergy.
A cell 524b contains binary data indicating whether the EMR indicates that the patient has a gluten allergy. In this example, the value of 1 indicates that the patient does have a gluten allergy.
A cell 524c contains integer data representing the patient's weight in kilograms, as indicated by the EMR. In this example, the value of 80 indicates that the patient weighs 80 kilograms.
A set of cells 524d, 524e, and 524f encoding information about a prescription of 250 mg of Aspirin to the patient, as indicated by the EMR. In this example, cell 524d contains a value of 1, representing the active ingredient, acetylsalicylic acid; cell 524e contains a value of 1, representing the dose form of the aspirin (e.g., spray, fluid, or tablet); and cell 524f contains a value of 250, representing the number of milligrams in the prescription.

The parameters and parameter values illustrated in FIG. 5 are merely examples and do not constitute limitations of the present invention. In practice, data in the saved initial EMR state may contain any number and variety of parameters having any values. Such parameters may be encoded in ways other than in the manner illustrated in FIG. 5.

As illustrated in FIG. 5, the encoding 522 of the saved initial EMR state 156 may be compressed, such as by using an autoencoder, and the resulting compressed version of the state may be provided as input to the machine learning module 306.

In the example of FIG. 5, the encoding 522 is used as the input vector to train the autoencoder, and an encoding 502 is used as an output vector to train the autoencoder. Note that the output vector 502 has the same contents as the input vector 522 (i.e., cells 504a-f of the output vector 502 contain the same data as the corresponding cells 524a-f of the input vector 522). A hidden layer 512 has a lower dimension (e.g., fewer cells) than the input vector 522 and the output vector 502. In the particular example of FIG. 5, the input vector 522 contains six cells 524a-f and the hidden layer 512 contains three cells 514a-c, but this is merely an example.

The autoencoder may be executed to learn how to reproduce the input vector 522 by learning a lower-dimensional representation in the hidden layer 512. The result of executing the autoencoder is to populate the cells of the hidden layer 512 with data which represent the data in the cells 524a-f of the input layer 522 in compressed form. The resulting hidden layer 512 may then be used as an input to the machine learning module 306 instead of the saved initial EMR state 156. Similarly, the state differences 304 may be encoded with an autoencoder, and its hidden layer 512 may be passed as the target (i.e. the expected outcome) to the machine learning module 306.

The example of FIG. 5 represents an encoding of discrete data. The saved initial EMR state 156 and/or saved final EMR state 160 may also contain non-discrete data, such as text blobs (e.g., clinical narratives) that were entered by a physician, such as by typing. Text blobs do not have an upper bound on their length, which complicates their encoding. Embodiments of the present invention may, for example use sequence-to-sequence models to condense the narrative into a finite vector. Such a model may learn how to map an input sequence to itself. A finite hidden representation may then be used as the input to the machine learning module 306. Embodiments of the present invention may further improve such an encoding by using word embeddings. Such word embeddings may, for example, be trained independently on a large amount of clinical and non-clinical text, which may or may not require supervised data. Embodiments of the present invention may use a similar sequence-to-sequence modeling approach with appropriate word embeddings to encode the transcript 150.

Embodiments of the present invention have a variety of advantages. For example, as described above, scribes typically manually update the patient's EMR based on the physician-patient encounter. Doing so is tedious, time-consuming, and error-prone. Embodiments of the present invention address these shortcomings of existing techniques for updating the patient's EMR by learning how to automatically update the patient's EMR, and by then performing such automatic updating.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, the system 100 and method 200 use a signal processing module 120 to separate the physician speech 116a and the patient speech 116b from each other in the audio signal 110. Among other examples, the machine learning module 306 performs machine learning techniques which are inherently rooted in computer technology.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

1. A method performed by at least one computer processor executing computer program instructions tangibly stored on at least one non-transitory computer-readable medium, the method comprising, at a transcription job routing engine:
(A) saving an initial state of an electronic medical record (EMR) of a first person;
(B) saving a final state of the EMR of the first person after the EMR of the first person has been modified based on speech of the first person and speech of a second person;
(C) identifying differences between the initial state of the EMR of the third person and the final state of the EMR of the third person; and
(D) applying a machine learning module to: (1) a transcript of the speech of the first person and the speech of the second person; and (2) the differences between the initial state of the EMR of the first person and the final state of the EMR of the first person, to generate a mapping between: (a) the transcript of the speech of the first person and the speech of the second person; and (b) the differences between the initial state of the EMR and the final state of the EMR.

2. The method of claim 1, further comprising, before (B):
(E) capturing the speech of the first person and the speech of a second person to produce at least one audio signal representing the speech of the first person and the speech of the second person; and
(F) applying automatic speech recognition to the at least one audio signal to produce the transcript of the speech of the first person and the speech of the second person.

3. The method of claim 2, further comprising, before (B):
(G) identifying an identity of the first person;
(H) identifying an identity of the second person; and
wherein (F) comprises producing the transcript of the speech of the first person and the speech of the second person based on the identity of the first person, the identity of the second person, and the speech of the first person and the speech of the second person.

4. The method of claim 3, wherein (F) further comprises associating the identity of the first person with a first portion of the transcript and associating the identity of the second person with a second portion of the transcript.

5. The method of claim 1, wherein (A) comprises converting the initial state of the EMR into a text file.

6. The method of claim 1, wherein (A) comprises converting the initial state of the EMR of the first person into a list of discrete medical domain model instances.

7. The method of claim 1, wherein (B) comprises converting the final state of the EMR of the first person into a text file.

8. The method of claim 1, wherein (B) comprises converting the final state of the EMR of the first person into a list of discrete medical domain model instances.

9. The method of claim 1, wherein (C) comprises using non-linear alignment techniques to identify the differences between the initial state of the EMR of the first person and the final state of the EMR of the first person.

10. The method of claim 1, further comprising:
(E) saving an initial state of an electronic medical record (EMR) of a third person;
(F) saving a final state of the EMR of the third person after the EMR of the third person has been modified based on speech of the third person and speech of a fourth person;
(G) identifying differences between the initial state of the EMR of the third person and the final state of the EMR of the third person; and
(H) applying the machine learning module to:
(1) the transcript of the speech of the first person and the speech of the second person;
(2) the differences between the initial state of the EMR of the first person and the final state of the EMR of the first person;
(3) the transcript of the speech of the third person and the speech of the fourth person;
(4) the differences between the initial state of the EMR of the third person and the final state of the EMR of the third person;
thereby generating a mapping between text and EMR state differences.

11. A system comprising at least one non-transitory computer-readable medium having computer program instructions stored thereon for causing at least one computer processor to perform a method, the method comprising, at a transcription job routing engine:
(A) saving an initial state of an electronic medical record (EMR) of a first person;
(B) saving a final state of the EMR of the first person after the EMR of the first person has been modified based on speech of the first person and speech of a second person;
(C) identifying differences between the initial state of the EMR of the third person and the final state of the EMR of the third person; and
(D) applying a machine learning module to: (1) a transcript of the speech of the first person and the speech of the second person; and (2) the differences between the initial state of the EMR of the first person and the final state of the EMR of the first person, to generate a mapping between: (a) the transcript of the speech of the first person and the speech of the second person; and (b) the differences between the initial state of the EMR and the final state of the EMR.

12. The system of claim 11, wherein the method further comprises, before (B):
(E) capturing the speech of the first person and the speech of a second person to produce at least one audio signal representing the speech of the first person and the speech of the second person; and
(F) applying automatic speech recognition to the at least one audio signal to produce the transcript of the speech of the first person and the speech of the second person.

13. The system of claim 12, wherein the method further comprises, before (B):
(G) identifying an identity of the first person;
(H) identifying an identity of the second person; and
wherein (F) comprises producing the transcript of the speech of the first person and the speech of the second person based on the identity of the first person, the identity of the second person, and the speech of the first person and the speech of the second person.

14. The system of claim 13, wherein (F) further comprises associating the identity of the first person with a first portion of the transcript and associating the identity of the second person with a second portion of the transcript.

15. The system of claim 11, wherein (A) comprises converting the initial state of the EMR into a text file.

16. The system of claim 11, wherein (A) comprises converting the initial state of the EMR of the first person into a list of discrete medical domain model instances.

17. The system of claim 11, wherein (B) comprises converting the final state of the EMR of the first person into a text file.

18. The system of claim 11, wherein (B) comprises converting the final state of the EMR of the first person into a list of discrete medical domain model instances.

19. The system of claim 11, wherein (C) comprises using non-linear alignment techniques to identify the differences between the initial state of the EMR of the first person and the final state of the EMR of the first person.

20. The system of claim 11, wherein the method further comprises:
(E) saving an initial state of an electronic medical record (EMR) of a third person;
(F) saving a final state of the EMR of the third person after the EMR of the third person has been modified based on speech of the third person and speech of a fourth person;
(G) identifying differences between the initial state of the EMR of the third person and the final state of the EMR of the third person; and
(H) applying the machine learning module to:
(1) the transcript of the speech of the first person and the speech of the second person;
(2) the differences between the initial state of the EMR of the first person and the final state of the EMR of the first person;
(3) the transcript of the speech of the third person and the speech of the fourth person;
(4) the differences between the initial state of the EMR of the third person and the final state of the EMR of the third person;
thereby generating a mapping between text and EMR state differences.
